(19) United States
(12) Patent Application Publication
(10) Pub. No.: US 2018/0185378 A1
(43) Pub. Date: Jul. 5, 2018

(54) ANTIMICROBIAL DELIVERY SYSTEM FOR THE PREVENTION AND TREATMENT OF INFECTIONS IN THE COLON

(71) Applicant: Cormedix Inc., Berkeley Heights, NJ (US)

(72) Inventor: Robert DiLuccio, Haymarket, VA (US)

(21) Appl. No.: 15/861,248

(22) Filed: Jan. 3, 2018

Related U.S. Application Data
(60) Provisional application No. 62/442,778, filed on Jan. 5, 2017.

(51) Int. Cl. A61K 31/541; C07D 285/18; A61K 9/20; A61K 9/00; A61P 1/00

(52) U.S. Cl. CPC A61K 31/541; C07D 285/18; A61P 1/00; A61K 9/0053; A61K 9/2077

(57) ABSTRACT

This invention relates to the prophylactic and/or therapeutic application of antimicrobials that are, for example, administered orally as a delayed release formulation designed to release the drug to the distal small intestine and/or colon in high quantities and density, as a method for the prevention and/or treatment of infections in the colon.

### REFERENCE TO PENDING PRIOR PATENT APPLICATION

This patent application claims benefit of pending prior U.S. Provisional Patent Application Ser. No. 62/442,778, filed Jan. 5, 2017 by Cormedix, Inc. and Robert DiLuccio for ANTIMICROBIAL DELIVERY SYSTEM FOR THE PREVENTION AND TREATMENT OF INFECTIONS IN THE COLON (Attorney's Docket No. CORMEDIX-19 PROV), which patent application is hereby incorporated herein by reference.

### FIELD OF THE INVENTION

This invention relates to the prophylactic and/or therapeutic application of an antimicrobial that is, for example, administered orally as a delayed-release formulation designed to release the antimicrobial to the distal small intestine, proximal to the colon, or in the colon, in high quantities and density, whereby to provide a method for the prevention and/or treatment of, for example, colitis or Crohn's disease.

Among other things, the present invention relates to a method for treating a diseased state in the colon of a human, comprising enterally administering to the individual in need of such treatment an effective amount of a composition comprising 4,4′-methylenebis-(tetrahydro-1,2,4-thiadiazine)-1,1,1′,1′-tetraoxide, commonly known as taurolidine.

### BACKGROUND OF THE INVENTION

Bacteria are believed to “cross-talk” by the elaboration of certain chemicals which can up-regulate genes. These genes then, in their “switched-on state”, produce certain substances which give the bacteria an ability to exploit their environment. It is thought that these substances that allow “cross-talking” between bacterial cells to turn on these genes occurs when specific densities of bacteria are encountered. This required density effect is frequently referred to as “quorum sensing”.

It is the ability of “normal bacterial flora” (i.e., the bacteria that normally populate a healthy gastrointestinal tract, and particularly the colon, sometimes referred to as “the intestinal microbiome”) to resist and prevent this overgrowth of a single bacterial species which is essential to normal colonic health. While toxigenic microorganisms can be a normal component of one's bowel flora, their presence in low numbers precludes the ability to reach a critical number for quorum sensing to occur and thus are not able to cause toxin-related colitis.

However, once the normal flora is disrupted by antibiotics, the disruption of the microbiome in the colon creates an environment conducive to an overgrowth of pathogenic microorganisms that can result in a significant health hazard.

Crohn's disease, an idiopathic inflammatory bowel disease, is characterized by chronic inflammation at various sites in the gastrointestinal tract. While Crohn's disease most commonly affects the distal ileum and colon, it may manifest itself in any part of the gastrointestinal tract from the mouth to the anus and perianal area. Up to the present time, all drugs used in the treatment of Crohn's disease function as prophylactics against the symptoms of Crohn's disease (e.g., inflammation) as opposed to being curatives. Established chronic Crohn's disease is characterized by lifelong exacerbations.

Since its first documentation more than two hundred years ago, the incidence of Crohn's disease has increased markedly and universally throughout the world's populations. The disease is non-discriminating with respect to sex, showing relatively equal incidence of Crohn's disease in both sexes. The anatomic distribution of Crohn's disease has changed in recent years, with an increase in the incidence of large bowel disease. In addition, studies have shown that in the elderly, isolated colonic Crohn's disease is more common than in patients whose disease presents at a younger age, i.e., ages 60 and lower.

The etiology of Crohn's disease is unknown. Immunologic factors have been examined; possible infectious agents have included various enteric bacteria, viruses and chlamydiae. Dietary factors (e.g., a fiber-poor diet) and stress have also been considered. Mycobacteria is now receiving significant focus. The isolation of several different mycobacterial species from Crohn's disease patients, and the knowledge that M. paratuberculosis causes a granulomatous intestinal disease in ruminants, has generated keen interest in this organism.

Longstanding Crohn's disease predisposes patients to cancer, the most common type being adenocarcinoma which generally occurs in areas where Crohn's disease has been present for many years. Both small bowel and colon cancers occur with increased incidence in Crohn's disease patients. The increased cancer risk in Crohn's disease patients is most established for cases of cancer of the ileum, for which the relative risk in Crohn's disease is at least 100-fold greater than in an age/sex-matched control population.

### SUMMARY OF THE INVENTION

This invention relates to the delivery of an antimicrobial agent to the colonic flora as a method of treatment for, for example, colitis. Additionally, the present invention also relates to compositions and methods for the management of Crohn's disease. More particularly, the present invention relates to the administration of taurolidine to combat the inflammatory effects and bacterial causes of colitis or Crohn's disease.

This invention pertains to the prophylactic and/or therapeutic application of taurolidine that can be administered as an oral, delayed-release formulation designed to release its antimicrobial content just proximal to the colon, or in the colon, in high quantities and density. Such applications are intended to be used easily and repeatedly, daily and over extended periods of time. The taurolidine employed in the present invention can be formulated into capsules, tablets, caplets and/or other specialty-formulated oral delivery systems that will release their contents (comprising the antimicrobial taurolidine) in the distal small intestine and/or colon. Alternatively, the taurolidine may be administered directly to those locations as a suspension via a catheter or cannula if such access is readily available.

The present invention provides a method for the treatment of, or prevention of, a colitis infection in a patient in need of such treatment or prevention, by the administration of an antimicrobial (taurolidine), with or without intestinal motility agents, and combinations thereof; and a delivery vehicle, including an oral delivery vehicle wherein the oral delivery vehicle releases the taurolidine to at least the distal small intestine and/or colon of the individual.

The present invention further provides a method for the treatment of an individual wherein the treatment is prophylactic to prevent initial and/or recurrent or refractory colonic infection. The present invention further provides a method for the treatment of an individual wherein the treatment is prophylactic use applied in patients at risk of bacterial infection, wherein the patients are at risk due to antibiotic use, advanced age, co-morbidities, underlying disease severity, and/or exposure to other patients with colonic infections, and/or prolonged hospitalization.

The present invention further provides, among other things:
a method for treating a disease or condition in a patient by the use of microparticles that comprise taurolidine and have an immediate-release coating, or a delayed-release coating, or are a combination of immediate- and delayed-release microparticles; and/or
a method wherein the coated (immediate- and/or delayed-release) microparticles are mixed as a suspension and administered orally; and/or
a method wherein the dosage form is selected from the group consisting of a pre-filled syringe, a user-filled syringe, a sachet, a large capsule, and a squeezable tube that will allow administration of the antimicrobial dosage form through a tube or catheter; and/or
a method wherein the administration of the antimicrobial dosage to the bowel is through a stoma or rectally; and/or
a method wherein the antimicrobial dosage form protects the activity of the taurolidine during storage and delivery and until its release in the distal small intestine and/or colon; and/or
a method wherein the taurolidine delivery system is administered with a regimen selected from the group consisting of once a day and multiple times a day, with food, without food, and combinations thereof.

The antimicrobial dosage form may contain intestinal motility agents, and combinations thereof; and a delivery vehicle, including an oral delivery vehicle wherein the oral delivery vehicle releases the taurolidine to the distal small intestine and/or colon of the intestine of the individual.

The invention further provides, among other things:
an oral delivery system (also sometimes referred to herein as a delivery vehicle) for the taurolidine, wherein:
   the delivery vehicle is selected from the group consisting of pills, tablets, caplets, capsules, soft gels, and coated microparticles, that will release the taurolidine agent in the distal small intestine and/or colon; and/or
   the taurolidine agent is present, and is in a dosage form selected from immediate-release, delayed-release and/or extended-release, which is released in the distal small intestine and/or colon, and targeted release which is targeted to be released in the distal small intestine and/or colon (i.e., the delivery vehicle shields the taurolidine from premature hydrolysis prior to the taurolidine reaching its intended target site); and/or
an oral delivery system wherein the delivery vehicle is targeted to release the taurolidine agent in the distal small intestine and/or colon of the individual; and/or
an oral delivery system wherein the coated microparticles:
   are in a sachet; and/or
   are selected from the group consisting of microparticles with an immediate-release coating, microparticles with a delayed-release coating, a microencapsulated antimicrobial, and combinations thereof;
   can be applied directly to food as a sprinkle; and/or
   are mixed as a suspension and administered directly to the intestine, optionally through a catheter or cannula; and/or
   are in a capsule, which optionally has a coating selected from immediate-release, delayed-release, and extended-release, which is released in the distal small intestine and/or colon, and targeted-release which is targeted to be released in the distal small intestine and/or colon; and/or
an oral delivery system for delivering tauroldine, wherein the oral delivery system comprises an oral delivery vehicle which:
   is coated so as to ensure the delayed release of its content and will offer required shelf-life stability; and/or
   is a capsule, wherein the capsule comprises materials selected from the group consisting of hydroxyl-propyl methylcellulose, gelatin, starch, and combinations thereof; and/or
   has a pH-dependent coating; and/or
   is coated with a copolymer selected from the group consisting of methacrylic acid, methacrylates, and combinations thereof that dissolve at pH 5.5 to 7.0, and may be used to achieve distal small intestinal and/or colonic delivery and is time-dependent; and/or
   wherein the thickness of the coating constitutes an additional factor that can be employed to extend the release of the active ingredient (i.e., taurolidine); and/or
   is an oral delivery system wherein the delivery vehicle has a delayed delivery using a hydrogel plug; and/or
   is an oral delivery system wherein the delivery vehicle's time-dependent delivery systems utilize ethylcellulose as the release-determining polymer; and/or
   is an oral delivery system wherein the delivery vehicle has delayed delivery based on a combination of pH-dependent and time-dependent systems; and/or
   has delayed delivery based on bacterial degradation of the formulation coating.

In one preferred form of the invention, there is provided a method for treating a condition in the gastrointestinal tract of a patient, the method comprising:
administering a therapeutically-effective amount of a composition comprising taurolidine to the distal small intestine or the colon of the patient.

In another preferred form of the invention, there is provided a composition for oral administration to a patient for treating a condition in the gastrointestinal tract of toe patient, the composition comprising a taurolidine core contained within a decayable delivery vehicle, wherein the decayable delivery vehicle shields the taurolidine core from premature hydrolyzation of the taurolidine as the composition moves from the mouth of the patient to the distal small intestine or the colon of the patient and releases the taurolidine for hydrolyzation in the distal small intestine or the colon of the patient.

### DETAILED DESCRIPTION OF THE PREFERRED EMBODIMENTS

Taurolidine occurs as a white to off-white powder having the molecular formula C₇H₁₆N₄O₄S₂.

Taurolidine's general characteristics include acceptable stability in the solid state when stored at ambient conditions, melting with decomposition at approximately 170° C., and the following solubility in aqueous solutions and organic solvents:

| | |
|---|---|
| Water | 1% at 20° C. |
| Dilute HCl | soluble |
| Dilute NaOH | soluble |
| CHCl₃ | insoluble |
| EtOH | sparingly soluble |
| DMF | 1 g in 2 mL/ca. 60° C. |
| Acetone | 1 g in 120 mL/Boiling |
| Ethanol | 1 g in 130 mL/Boiling |
| Methanol | 1 g in 170 mL/Boiling |
| Ethyl Acetate | 1 g in 200 mL/Boiling |

A saturated solution of taurolidine in deionized water has a pH of 7.4. The apparent partition coefficient of taurolidine between octanol and water (buffered at pH 7.2) is approximately 0.13 and would therefore not be predicted to accumulate to any significant extent in fatty tissues.

The synthesis of taurolidine is covered in several patents including U.S. Pat. No. 3,423,408; Switzerland Pat. No. 482,713 and United Kingdom Pat. No. 1,124,265, and is carried out in five stages:
(i) potassium phthalimidoethane sulphonate is prepared from taurinc, phthalic anhydride, glacial acetic acid and potassium acetate;
(ii) potassium phthalimidoethane sulphonate is then converted to phthalimidoethane sulphonylchloride by chlorination with phosphorous oxychloride;
(iii) phthalimidoethane sulphonylchloride is reacted with ammonia to form phthalimidoethane sulphonamide;
(iv) phthalimidoethane sulphonylchloride is reacted with hydrazine hydrate and in the subsequent hydrazinolysis forms taurinamide hydrochloride; and
(v) taurolidine is prepared from taurinamide hydrochloride and formaldehyde.

The actions of taurolidine have been described in U.S. patent application Ser. No. 09/151,885 filed Sep. 11, 1998, and in U.S. Pat. No. 3,423,408, and elsewhere in the literature. In addition, the following United States patents describe various uses for, and compositions containing, taurolidine: U.S. Pat. No. 4,107,305, treatment of endotoxaemia; U.S. Pat. No. 4,337,251, elimination of adhesion formation as a result of surgery; U.S. Pat. No. 4,587,268, resorbable aqueous gels; U.S. Pat. No. 4,604,391, prevention of the occurrence of osteitis or osteomyelitis; U.S. Pat. No. 4,626,536, combating toxic proteins or peptides in the blood; U.S. Pat. No. 4,772,468, treatment of bone cavities; and U.S. Pat. No. 4,882,149, directed to methods for filling congenital, surgical or traumatic defects with compositions comprising natural bone mineral having absorbed therein/thereon taurolidine.

Taurolidine's mechanism of action, unlike that of known antibiotics, is based on a chemical reaction. While not being bound by any theory, during the metabolism of taurolidine to taurinamide and ultimately taurine and water, methylol groups are liberated which chemically react with the mureins in bacterial cell walls, which results in the denaturing of the complex polysaccharide and liposaccharide components of the bacterial cell walls as well as changing the double-stranded DNA of the plasmid to a denatured or single-stranded. DNA. Thus, the taurolidine hydrolyzes in the body to form methylol groups which then attack the cell walls of the bacteria, whereby to kill the bacteria.

Taurolidine has been shown to be safe and well tolerated at systemic doses exceeding 40 g/day and cumulative doses up to, and exceeding, 300 g/day.

It has long been the goal of the pharmaceutical industry to produce antibiotic medicinal substances that have the power to kill, or at least to arrest the growth of, many disease-causing mycobacteria such as those associated with Crohn's disease.

In general, the compositions of the present invention can be readily utilized in a variety of pharmaceutical formulations, preferably formulations which release taurolidine in the gut. The disclosed medicament may be used alone of in combination with a pharmacologically and/or nutritionally acceptable carrier.

When the taurolidine compositions of the present invention are provided in solution form, the formulations of taurolidine generally utilized are sterile solutions containing about 0.5%, 1.0%, 2.0% or about 4.0% taurolidine by weight in water.

The compositions of the present invention for the management of Crohn's disease may take any of a variety of forms as noted above, however, in terms of the ability to deliver the active material taurolidine to the target site of action, i.e., the gastrointestinal tract, it is preferred to use enema, suppository, tablet, capsule, solution or suspension formulations. A particularly preferred form of the present invention is a delayed- or sustained-release form which coats microgranules of taurolidine with a semipermeable membrane such as ethyl cellulose for a gradual pH-dependent release throughout the gut, whereby to delay hydrolysis of the taurolidine into its active methylol groups until the taurolidine has been delivered to the target region.

Solid carriers and diluents suitable for use include sugars such as lactose and sucrose; cellulose derivatives such as carboxymethyl cellulose, ethylcellulose, methylcellulose, etc.; gelatin including hard and soft gelatin capsules; talc, cornstarch; stearic acid; and magnesium stearate.

Suspension formulations may additionally contain benzoic acid, coloring, natural and artificial flavors, glycerin, kaolin, magnesium, aluminum silicate, methyl paraben, pectin, purified water, saccharin, sodium hydroxide and sucrose.

The percentage of taurolidine in the composition can be varied over wide ranges, and the quantity of medicament furnished by each individual tablet, capsule, solution or suspension is relatively unimportant since the indicated daily dose can be reached by administering either one or a plurality of tablets, capsules, solutions or suspensions.

In one form of the invention, the composition comprises a therapeutically-effective amount of taurolidine and a suitable carrier which shields the taurolidine from premature hydrolysis and then releases the taurolidine for hydrolyzation at the target region. In this form of the invention, the suitable carrier may comprise a carrier that can be triggered to release the drug at the desired region in the colon. By way of example but not limitation, such a carrier may comprise a poloxamer composed of PEG (polyethylene glycol) and polypropylene glycol which acts as a non-ionic surfactant but can be made at a high enough molecular weight (>1000) to form a waxy to firmer polymer.

In another form of the invention, the composition comprises a therapeutically-effective amount of taurolidine and a hydrogel which shields the taurolidine from premature hydrolysis and then releases the taurolidine for hydrolyzation at the target region. In a preferred form of the invention, the hydrogel comprises a hydrolysable segment sensitive to serine proteases in the colon. By way of example but not limitation, such a hydrogel may comprise poloxamers such as Poloxamer 188 or Poloxamer 407.

In yet another form of the invention, the composition comprises a therapeutically-effective amount of taurolidine in crystallized salt form and a suitable carrier (e.g., a hydrogel where the salt is suspended in a gel such as Poloxamer 188, or a solution where the salt is dispersed in solution such as a solution containing 0.5%-4% taurolidine by weight in water).

The formulation used for administration of taurolidine may be specialty coated to meet the requirements of the present invention, i.e., the taurolidine must be released in the distal small intestine and/or colon. The material used for manufacturing of capsules is such that it is amenable to coating that will ensure the delayed release of its content and will offer extended shelf storage stability. A preferred material used for manufacturing of capsules that will meet the requirements of the present invention is hydroxy-propyl-methyl-cellulose (U.S. Pat. No. 7,094,425), although other materials may be used as well, e.g., gelatin, starch, etc. Specialty coatings for such a delayed-release formulation may be achieved using different principles. The most convenient is the pH-dependent coating. Coatings utilizing copolymers consisting of methacrylic acid and methacrylates that preferably dissolve at pH 5.5 to 7.0 may be used to achieve distal small intestinal and colonic delivery of the taurolidine. The thickness of the coating constitutes an additional factor that can be employed to adjust the time required for disintegration of the coated formulation. It has been shown that hydroxypropylmethyl cellulose (HPMC) capsules coated with the polymer Eudragit® FS30D release their contents in the distal small intestine and primarily in the proximal colon. Another example is a coating utilizing Eudragit LI 00 and Eudragit S I 00 at an 80:20 ratio. Starch seamless capsules so coated have achieved distal ileal and colonic delivery (U.S. Pat. No. 6,228,396 B1). The pre-coating of capsules for delayed release prior to filling with the antimicrobial taurolidine may be the preferred method that will further reduce a damaging effect of the antimicrobial content (i.e., taurolidine) and will ensure the tight and stable closure of capsules. Some other principles for achieving distal small intestinal and/or colonic delivery may utilize dissolution of a carrier that is time-dependent. Examples of such delayed delivery are formulations using a hydrogel plug such as PULSINCAP™ (International Patent Publication No. WO 90/09168). Other time-dependent delivery systems utilize ethylcellulose as the release-determining polymer. Other examples of delayed-release systems are the TIMECLOC® system and the Time-Controlled Explosion (TES) system. Also, a combination of pH-dependent and time-dependent systems can be utilized. Some of the colonic targeted delivery systems are based on bacterial degradation of the formulation coating. An example of this is the degradation of polymers containing azo bonds that are cleaved by the colonic bacteria azo reductase enzyme. Various polymers of such type have been described in the literature. Other polymers can also be utilized to achieve colonic delivery (U.S. Pat. No. 6,368,629 B1).

Antimicrobial Dosage Forms

The compositions of the present invention can be provided in the form of a microcapsule, a capsule, a tablet, an implant, a troche, a lozenge (mini-tablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip, a sachet, etc. Compositions can also be administered after being mixed with, for example, yogurt or fruit juice and swallowed, or followed with a drink or beverage. These forms are well known in the art of medication delivery and are packaged appropriately. The compositions can be formulated for oral or rectal delivery.

The compositions of the present invention can be coated with one or more enteric coatings, seal coatings, film coatings, barrier coatings, compress coatings, fast disintegrating coatings, enzyme degradable coatings, etc. Multiple coatings can be applied for desired performance. Further, the antimicrobial dosage form can be designed for immediate release, pulsatile release, controlled release, extended release, delayed release, targeted release, synchronized release, or targeted delayed release. For release control, solid carriers can be made of various component types and levels or thicknesses of coats, with or without an active ingredient. Such diverse solid carriers can be blended in a dosage form to achieve a desired performance. The definitions of these terms are known to those skilled in the art. In addition, the antimicrobial dosage form release profile can be affected by a polymeric matrix composition, a coated matrix composition, a multi-particulate composition, a coated multi-particulate composition, an ion-exchange resin-based composition, an osmosis-based composition, a biodegradable polymeric composition, etc. without wishing to be bound by theory, it is believed that the release may be effected through favorable diffusion, dissolution, erosion, ion-exchange, osmosis or combinations thereof.

When formulated as a capsule, the capsule can be a hard or soft gelatin capsule, a starch capsule, or a cellulosic capsule. Although not limited to capsules, such antimicrobial dosage forms can further be coated with, for example, a seal coating, an enteric coating, an extended release coating, or a targeted delayed release coating. These various coatings are well known in the art of drug delivery, but for clarity, the following brief descriptions are provided below.

Seal coating, or coating with isolation layers: thin layers of up to 20 microns in thickness can be applied for a variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or ELDRAGIT® E 100, may be particularly suitable for taste-masking applications. Traditional enteric coating materials listed elsewhere can also be applied to form an isolating layer.

Extended or delayed release coatings are designed to effect delivery over an extended period. The extended or delayed release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended or delayed release dosage forms can be readily designed by one skilled in art to achieve delivery to both the small and large intestines, to only the small intestine, or to only the large intestine, depending upon the choice of coating materials and/or coating thickness.

Enteric coatings are mixtures of acceptable excipients which are applied to, combined with, mixed with or otherwise added to the carrier or composition. The coating may be applied to a compressed or molded or extruded tablet, a gelatin capsule, and/or pellets, heads, granules or particles of the carrier or composition. The coating may be applied through an aqueous dispersion or after dissolving in an appropriate solvent. Additional additives and their levels, and selection of a primary coating material or materials, will depend on the following properties:
1. resistance to dissolution and disintegration in the stomach;
2. impermeability to gastric fluids while in the stomach;
3. ability to dissolve or disintegrate rapidly at the target intestine site;
4. physical and chemical stability during storage;
5. non-toxicity;
6. easy application as a coating (substrate friendly); and
7. economical practicality.

Antimicrobial dosage forms of the compositions of the present invention can also be formulated as enteric coated, delayed release oral dosage forms, e.g., as an oral dosage form of a composition as described herein which utilizes an enteric coating to affect release in the lower gastrointestinal tract. The enteric coated dosage form may be a compressed or molded or extruded tablet (coated or uncoated) containing granules, pellets, beads or particles of taurolidine and/or other composition components, which may be themselves coated or uncoated. The enteric coated oral dosage form may also be a capsule (coated or uncoated) containing pellets, beads or granules of the solid carrier or the composition, which are themselves coated or uncoated. Delayed release generally refers to the delivery so that the release can be accomplished at some generally predictable location in the lower intestinal tract more distal to that which would have been accomplished if there had been no delayed release. The preferred method for delay of release is coating. Any coatings should be applied to a sufficient thickness such that the entire coating does not dissolve in the gastrointestinal fluids at a pH below about 5, but does dissolve at a pH of about 5 and above. It is expected that any anionic polymer exhibiting a pH-dependent solubility profile can be used as an enteric coating in the practice of the present invention to achieve delivery to the lower gastrointestinal tract. Polymers for use in the present invention are preferably anionic carboxylic polymers.

Shellac, also called purified lac, is a refined product obtained from the resinous secretion of an insect. This coating dissolves in media of pH>7.

Colorants, detackifiers, surfactants, antifoaming agents, lubricants, stabilizers such as hydroxypropyl cellulose, and acids/bases may be added to the coatings in addition to plasticizers to solubilise or disperse the coating material, and to improve coating performance and the coated product.

In carrying out the method of the present invention, the composition of the invention may be administered to mammalian species (such as dogs, cats, humans, etc.), and as such may be incorporated in a conventional systemic dosage form (such as a tablet, capsule or elixir). The above dosage forms will also include the necessary carrier material, excipient, lubricant, buffer, antibacterial, bulking agent (such as mannitol), anti-oxidants (ascorbic acid or sodium bisulfite) or the like.

The dose administered may be adjusted according to the age, weight and condition of the patient, as well as the route of administration, dosage form and regimen and the desired result.

The compositions of the invention may be administered in single or divided doses of one to four times daily, or may be administered multiple times per day. It may be advisable to start a patient on a low dose and work up gradually to a higher dose.

Tablets of various sizes can be prepared, e.g., or about 2 to 2000 mg in total weight, containing the active ingredient (i.e., taurolidine), with the remainder being a physiologically acceptable carrier of other materials per accepted practice. Gelatin capsules can be similarly formulated. Liquid formulations can also be prepared by dissolving or suspending one or the combination of active substances in a conventional liquid vehicle acceptable for administration to provide the desired dosage in, for example, one to four teaspoons.

Antimicrobial dosage forms can be administered to the patient on a regimen of, for example, one, two, or other multiple doses per day.

To more finely regulate the dosage schedule, the active substance (i.e., taurolidine) may be administered separately in individual dosage units at the same time or carefully coordinated times. The respective substances can be individually formulated in separate snit dosage forms in a manner like that described above.

In formulating the compositions of the present invention, the active substance (i.e., taurolidine), in the amounts described above, may be compounded per accepted practice with a physiologically acceptable vehicle, carrier, excipient, binder, preservative, stabilizer, flavor, etc., in the unit dosage form.

### Examples

An example of an antimicrobial dosage form for the targeted delivery of taurolidine is a capsule containing the taurolidine. It is specialty coated to achieve delayed targeted release. The coating method in this example is pH-dependent that is most economical, easy to apply and offers flexibility as to the desired release characteristics.

Vcaps (Capsugel) made of EPMC material are selected for the delayed release of the antimicrobial (i.e., taurolidine) formulation. The taurolidine is disposed within the capsule, e.g., in a solution containing 1.5% taurolidine by weight in water. Vcaps has an advantage of easy coating due to the matte surface of the capsules. The coating used is configured for delayed-release of the taurolidine in the distal small intestine and colon.

For distal small intestinal delivery, the material used for coating is EUDRAGIT® 30 D-55 (Evonic Industries, Darmstadt, Germany) which dissolves at a pH of 5.5-6.0. The mean time for complete disintegration of capsules coated with Eudragit L30 D-55 was 2.4 hours in the in vivo tests. An alternative coating can be done utilizing Eudragit L30 D-50, which has similar dissolution characteristics (U.S. Pat. No. 7,094,425 B2). The preferred thickness of the coating is 8 mg/cm², although further adjustments to increase the time to complete disintegration of capsules can be made by increasing the thickness of the coating up to 15 mg/cm².

For colonic delivery, a coating with EUDRAGIT® FS 30D (Evonic Industries, Darmstadt, Germany) can be used which dissolves at a pH of 7.0 and higher. The in vivo tests showed complete disintegration of capsules with thickness of the coating of 6 mg/cm² at mean 6.9 hours. Similar results were reported for the in vitro testing (U.S. Pat. No. 7,094,425 B2).

An alternative coating method for colonic delivery utilizes a 3:1 mixture of EUDRAGIT® LI00 and Eudragit SI00 (U.S. Pat. No. 6,228,396 B1). Starch capsules so coated were shown in the in vivo testing to disintegrate primarily in the colon with mean time to dissolution of 6.0 hours.

An alternative coating method is to create separately the inner and the outer coat using EUDRAGIT®, polymers (U.S. Pat. No. 5,686,105).

### Additional Examples

(1) Oral Delivery

(a) Capsules

Capsules containing a therapeutically-effective amount of taurolidine may be ingested for delivery of the taurolidine to the distal small intestine or colon of a patient in order to prevent or treat gastrointestinal conditions such as general infections, colitis, Crohn's disease, etc. In this form of the invention, the capsules serve to shield the hydrolysable taurolidine from premature hydrolysis prior to the taurolidine reaching its intended target (i.e., the tissue of the distal small intestine or colon of the patient), and then the capsules decay so as to release the taurolidine for hydrolysis at the target site, whereby to form the methylol groups which then attack the cell walls of bacteria, whereby to kill the bacteria, and/or to otherwise treat the tissue of the patient.

The taurolidine contained within the capsules may be in crystalline form (e.g., a crystallized salt form of taurolidine); or the taurolidine may be in solution form (e.g., 0.5%-4% taurolidine by weight in water); or the taurolidine may be in mixture form (e.g., taurolidine mixed with a hydrolizable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc.); or the taurolidine may be in microparticle form (e.g., a core of hydrolysable taurolidine encapsulated by a hydrolysable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc.); or the taurolidine may be born by a carrier (e.g., propylene glycol, polyethylene glycol, petrolatum, glycerin, polyvinylpyrolidone, hyaluronic acid, etc.).

It should be appreciated that where the taurolidine is mixed with, or encapsulated by, a hydrolizable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc., the hydrolysable lipophilic material may facilitate passage of the mixture, or the microparticles, into the tissue of the patient, whereby to facilitate deeper penetration of the taurolidine before the taurolidine hydrolyzes into its active moieties (i.e., methylol groups).

The capsules themselves (i.e., the decayable shell containing the taurolidine) comprise a material which shields the “passenger” hydrolysable taurolidine as the capsules move from the mouth of the patient to the distal small intestine or colon of the patient, whereupon the capsules decay so as to release the taurolidine contents of the capsules. As noted above, the capsules may be formed of different materials which use different mechanisms for capsule decay, e.g., the capsules may comprise a time-dependent coating, a pH-dependent coating, etc.

(b) Encapsulated

If desired, the hydrolysable taurolidine may be encapsulated in a hydrolysable lipophilic excipient, i.e., so as to form microparticles. More particularly, in this form of the invention, a hydrolysable taurolidine core is covered by a hydrolysable lipophilic excipient, with the hydrolysable lipophilic excipient hydrolyzing as the encapsulated taurolidine moves from the mouth of the patient to the target region, whereby to shield the hydrolysable taurolidine from premature hydrolysis prior to reaching its intended target (i.e., the tissue of the distal small intestine or colon of the patient), and then releasing the taurolidine for hydrolysis at the target site, whereby to form the methylol groups which then attack the cell walls of bacteria, whereby to kill the bacteria, and/or to otherwise treat the tissue of the patient. By way of example but not limitation, the hydrolysable lipophilic excipient may comprise a saturated fatty alcohol or fatty acid of 8-15 carbon atoms or an unsaturated fatty alcohol or fatty acid of 8-18 carbon atoms, e.g., myristic acid or myristyl alcohol, etc.).

(2) Rectal Delivery

(a) Solution Or Suspension

A solution or suspension containing a therapeutically-effective amount of taurolidine may be delivered via the rectum to the distal small intestine or colon of a patient in order to prevent or treat gastrointestinal conditions such as general infections, colitis, Crohn's disease, etc. In this form of the invention, the solution or suspension carries the taurolidine which then hydrolyzes at the target site, whereby to form the methylol groups which then attack the cell walls of bacteria, whereby to kill the bacteria, and/or to otherwise treat the tissue of the patient. By way of example but not limitation, the solution may comprise 0.5%-4% taurolidine by weight in water.

(b) Suppository

A suppository comprising a therapeutically-effective amount of taurolidine may be manually delivered via the rectum to the target site (e.g., the distal small intestine or colon of a patient) so that the taurolidine hydrolyzes at the target site, whereby to form the active moieties (i.e., the methylol groups). The suppository may be in capsule form (where the capsule shell decays to release the “passenger” taurolidine for hydrolysis) or some other form, e.g., in mixture form (e.g., taurolidine mixed with a hydrolizable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc.); or the taurolidine may be in microparticle form (e.g., a core of hydrolysable taurolidine encapsulated by a hydrolysable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc.); or the taurolidine may be born by a carrier (e.g., propylene glycol, polyethylene glycol, petrolatum, glycerin, polyvinylpyrolidone, hyaluronic acid, etc.).

It should be appreciated that where the taurolidine is mixed with, or encapsulated by, a hydrolizable lipophilic material such as a saturated fatty alcohol or fatty acid of 8-15 carbon atoms or an unsaturated fatty alcohol or fatty acid of 8-18 carbon atoms, e.g., myristic acid or myristyl alcohol, etc., the hydrolysable lipophilic material may facilitate passage of the mixture, or the microparticle, into the tissue of the patient, whereby to facilitate deeper penetration of the taurolidine before the taurolidine hydrolysis into its active moieties (i.e., methylol groups).

### Modifications of the Preferred Embodiments

Many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for treating a condition in the gastrointestinal tract of a patient, the method comprising:
administering a therapeutically-effective amount of a composition comprising taurolidine to the distal small intestine or the colon of the patient.

2. A method according to claim 1 wherein the composition is administered to the patient orally.

3. A method according to claim 2 wherein the composition comprises a taurolidine core contained within a decayable delivery vehicle.

4. A method according to claim 3 wherein the decayable delivery vehicle shields the taurolidine core from premature hydrolyzation of the taurolidine as the composition moves from the mouth of the patient to the distal small intestine or the colon of the patient and releases the taurolidine for hydrolyzation in the distal small intestine or the colon of the patient.

5. A method according to claim 4 wherein the decay of the delivery vehicle occurs during transit from the mouth of the patient to the distal small intestine or the colon of the patient.

6. A method according to claim 4 wherein the decay of the delivery vehicle occurs in the distal small intestine or the colon of the patient.

7. A method according to claim 4 wherein the composition is in capsule form.

8. A method according to claim 4 wherein the composition is in tablet form.

9. A method according to claim 1 wherein the composition is administered to the patient rectally.

10. A method according to claim 9 wherein the composition is in suspension form.

11. A method according to claim 9 wherein the composition comprises taurolidine suspended in a gel.

13. A method according to claim 11 wherein the gel comprises a hydrogel.

14. A method according to claim 9 wherein the composition is in solution form.

15. A method according to claim 14 wherein the composition comprises 0.5%-4% taurolidine by weight in water.

16. A method according to claim 9 wherein the composition is in suppository form.

17. A method according to claim 16 wherein the composition comprises at least one from the group consisting of a capsule containing taurolidine and a gel containing taurolidine.

18. A composition for oral administration to a patient for treating a condition in the gastrointestinal tract of the patient, the composition comprising a taurolidine core contained within a decayable delivery vehicle, wherein the decayable delivery vehicle shields the taurolidine core from premature hydrolyzation of the taurolidine as the composition moves from the mouth of the patient to the distal small intestine or the colon of the patient and releases the taurolidine for hydrolyzation in the distal small intestine or the colon of the patient.

19. A composition according to claim 18 wherein the decay of the delivery vehicle occurs during transit from the mouth of the patient to the distal small intestine or the colon of the patient.

20. A composition according to claim 18 wherein the decay of the delivery vehicle occurs in the distal small intestine or the colon of the patient.

21. A composition according to claim 18 wherein the composition is in capsule form.

22. A composition according to claim 18 wherein the composition is in tablet form.
